(19) United States
(12) Patent Application Publication
(10) Pub. No.: US 2025/0000789 A1
(43) Pub. Date: Jan. 2, 2025

(54) SYSTEM FOR RELEASING A HAEMOSTATIC AGENT INTO THE UTERINE CAVITY

(71) Applicants: WOMED, MONTPELLIER (FR); CENTRE NATIONAL DE LA RECHERCHE SCIENTIFIQUE, PARIS (FR); UNIVERSITÉ DE MONTPELLIER, MONTPELLIER (FR); ECOLE NATIONALE SUPERIEURE DE CHIMIE DE MONTPELLIER, MONTPELLIER CEDEX 5 (FR)

(72) Inventors: XAVIER GARRIC, MONTPELLIER (FR); GONZAGUE ISSENMANN, PARIS (FR); ELODIE GATOUILLAT, MONTPELLIER (FR)

(21) Appl. No.: 18/698,799

(22) PCT Filed: Oct. 4, 2022
(86) PCT No.: PCT/EP2022/077602

§ 371 Date: Apr. 5, 2024

(30) Foreign Application Priority Data
Oct. 5, 2021 (FR) FR2110541

(51) Int. Cl. A61K 9/00; A61K 9/70; A61K 45/00; A61K 47/10; A61K 47/34

(52) U.S. Cl. CPC A61K 9/0039; A61K 9/70; A61K 45/00; A61K 47/10; A61K 47/34

(57) ABSTRACT

The subject of the invention is a degradable intrauterine system for the release of a haemostatic agent into the uterine cavity, comprising a degradable copolymer-based polymer matrix comprising at least one A and B block copolymer, and a haemostatic agent intended for release into the uterine cavity, wherein: block A is a polyester; block B is a poly(oxyethylene) (PEO); the weight-average molecular mass of the blocks B is a greater than or equal to 50 kDa; and the ethylene oxide unit/ester unit mole ratio is between 0.5 and 5. The invention also relates to a haemostatic agent for use in the treatment of uterine haemorrhage, wherein the haemostatic agent is delivered into the intrauterine cavity by means of a degradable intrauterine system according to the invention.

### TECHNICAL FIELD

The invention relates to a novel degradable intrauterine system for releasing a haemostatic agent into the uterine cavity

### Technological Background

Acute severe genital haemorrhage (or uterine haemorrhage), of uterine origin and unrelated to pregnancy, is excessive or prolonged bleeding of sufficient volume to require urgent intervention. It is common to classify such uterine bleeding according to its occurrence relative to menstruation. Menorrhagia is upper genital bleeding which coincides with menstruation but is abnormal as regards its abundance or duration. Metrorrhagia denotes any upper genital bleeding which occurs outside of menstruation. It may notably be caused by a pathology of the endometrium or myometrium (hyperplasia, cancer, polyp, fibroid, adenomyosis). Very heavy menorrhagia is an emergency problem and is considered as accidental metrorrhagia. Menometrorrhagia (i.e. the combination of menorrhagia and metrorrhagia) is bleeding from the uterus which is not caused by a tumour, an infection or pregnancy.

It is a common clinical problem and a source of distress for patients as it may be life-threatening. Uterine haemorrhage is, in fact, one of the main causes of maternal mortality. It is thus desirable to control such bleeding as soon as it arises.

The usual treatments for uterine haemorrhage in the emergency room are, in the first instance, venous injections of high-dose conjugated oestrogen, in order to regenerate the endometrium, covering the bare areas at the origin of the bleeding. Intravenous oestrogen alone can stop the bleeding but only five hours after the first administration. Conjugated oestrogens may cause nausea and vomiting. This is the only treatment specifically approved by the FDA for the treatment of acute severe uterine haemorrhage. Antifibrinolytics may also be prescribed and used, and are effective when given in time, within 3 hours of the onset of bleeding. The haemostatic effect generally occurs within 2-3 hours of administration. A rare but serious side effect is the possibility of secondary venous thrombosis. Another approach consists in performing tamponade haemostasis with devices that are usually in the form of a balloon. These devices are invasive and may be left in place for several hours, requiring the patient to remain in bed in the emergency room throughout the treatment. Removal of the balloon requires a further medical intervention for the patient.

There is thus a real need for an intrauterine system that can be easily positioned in the uterine cavity, which advantageously comes into contact with the uterine wall after swelling, and which allows the release of a haemostatic agent as quickly as possible on the uterine wall.

### SUMMARY OF THE INVENTION

In this context, the inventors have discovered that a haemostatic agent may be advantageously delivered into the uterine cavity by using as a vehicle copolymers based on blocks of polyesters, such as polylactic acid (PLA), and blocks of high molecular weight poly(oxyethylene) (PEO). Specifically, such copolymers make it possible to produce a material combining swelling and resorption properties that are particularly suitable for use in the uterine cavity to treat uterine bleeding. The inventors have thus developed a degradable intrauterine system from such a material also comprising a haemostatic agent. which in “dry” form has dimensions allowing easy placement from the uterine cervix, and which, once in the uterine cavity, moistens and unfolds to come into contact with the uterine wall and advantageously release the haemostatic agent directly onto the uterine wall. Specifically, the material according to the invention has a degree of swelling in an aqueous or humid medium allowing it to increase its volume up to about tenfold relative to the “dry” form. In addition, the material according to the invention allows a very rapid release of the haemostatic agent from the moment it is administered in the uterine cavity. Specifically, the release of the haemostatic agent can start, for example, about 30 seconds after administering the system into the uterine cavity. Notably, more than 60% of the haemostatic agent initially present in the material may, for example, be released within 10 minutes of its administration. Furthermore, the material of the intrauterine system preferably has anti-adhesion properties and the walls of the uterus are then kept separate by the intrauterine system, so that the cicatrization that follows haemostasis does not generate intrauterine adhesions or synechiae. Finally, the disintegration and evacuation time of the intrauterine system according to the invention is generally between 1 and 30 days, which not only allows the intrauterine system to remain in the uterine cavity for a sufficient period of time to treat the bleeding, but also to ensure that it is eliminated naturally, in particular before or during the next menstrual cycle.

One object of the invention is thus a degradable intrauterine system for the release of a haemostatic agent into the uterine cavity, comprising:
a degradable polymer matrix based on copolymers comprising at least one A and B block copolymer, wherein:
   block A is a polyester:
   block B is a poly(oxyethylene) (PEO):
   the weight-average molecular mass of the blocks B is greater than or equal to 50 kDa; and the ethylene oxide unit/ester unit mole ratio is between 0.5 and 5; and
at least one haemostatic agent intended to be released into the uterine cavity.

A subject of the invention is also a haemostatic agent for use in the treatment of uterine haemorrhage, wherein the haemostatic agent is delivered into the uterine cavity by means of a degradable intrauterine system according to the invention.

### BRIEF DESCRIPTION OF THE FIGURES

FIG. 1 is a schematic representation of a degradable intrauterine system according to the invention in the form of a trapezoidal film, which is particularly suitable for use in a human uterine cavity, and which is capable of coming into contact with the uterine wall after swelling and unfolding.

FIG. 2 is a schematic representation in longitudinal section of an example of an embodiment of the kit according to the invention comprising means for inserting a degradable intrauterine system according to the invention into the uterine cavity.

FIG. 3 shows the swelling of the degradable intrauterine system according to the invention in the form of a trapezoidal film. After 10 minutes, FIG. 3B (FIG. 3B) shows an increase in surface area of 79% relative to its initial surface area (FIG. 3A), and an increase in its mass (by water uptake) of 188% relative to its initial mass.

FIG. 4 shows the unfolding of a degradable intrauterine system according to the invention in the form of a trapezoidal film in a uterine cavity model, which is particularly suitable for use in a human uterine cavity, and which is capable of coming into contact with the uterine wall after swelling and unfolding.

FIG. 4A (FIG. 4A) shows the film immediately after insertion into the uterine cavity model, and FIG. 4B (FIG. 4B) shows the film unfolded 2 hours after insertion.

FIG. 5 shows the degradation of a degradable intrauterine system according to the invention in the form of a trapezoidal film in aqueous medium. The film remains in the form of a film for 5 days (FIG. 5A), then gradually degrades into fragments at 15 days (FIG. 5B) and dissolves at 30 days (FIG. 5C).

FIG. 6 shows the percentage of thrombin released in-vitro over time from a degradable intrauterine system according to the invention in trapezoidal film form (prototype A).

FIG. 7 shows the coagulation time (in minutes) obtained by means of a degradable intrauterine system according to the invention in trapezoidal film form.

FIG. 8 shows the evolution of the bleeding grade in an in vivo model (the pig) after insertion of films according to the invention (Prototypes B* and C) in comparison with a control test (no film inserted). A comparison between the initial bleeding grade and the bleeding grade at 6 minutes is performed to evaluate the hemostatic performance of the prototypes tested.

FIG. 9 shows the disintegration rate of films P3 and P4 according to the invention and comparative films Test 1, Test 2 and Test 3, after an incubation time in PBS between 0 and 12 minutes.

### DETAILED DESCRIPTION

The inventors have developed a degradable intrauterine system for the release of a haemostatic agent into the uterine cavity, which has mechanical and chemical properties that are particularly suitable for its use in the medical field, and in particular for the treatment of uterine haemorrhage. Specifically, the swelling and unfolding properties of the polymer composition used for forming the intrauterine system, combined with the haemostatic agent, make it possible to use it in the intrauterine cavity to treat uterine haemorrhage reliably and rapidly.

### The Degradable Intrauterine System

One subject of the present invention is a degradable intrauterine system for the release of a haemostatic agent into the uterine cavity, comprising:
a degradable polymer matrix based on copolymers comprising at least one A and B block copolymer, wherein:
   block A is a polyester:
   block B is a poly(oxyethylene) (PEO);
   the weight-average molecular mass of the blocks B is greater than or equal to 50 kDa; and
   the ethylene oxide unit/ester unit mole ratio is between 0.5 and 5, and
at least one haemostatic agent intended to be released into the uterine cavity.

In the context of the invention, the expression “polymer matrix based on” means a polymer matrix including the mixture and/or the product of the reaction between the base constituents used for the polymerization of this matrix, preferably only the product of the reaction between the various base constituents used for this matrix, some of which may be intended to react or likely to react with each other or with their immediate chemical environment, at least partly, during the various phases of the process of manufacturing the matrix, in particular during the polymerization step. Thus, the base constituents are the reagents intended to react together during the polymerization of the matrix.

In the context of the invention, the expression “between x and y” means that the values x and y are included.

According to the invention, the term “polyester” refers to any polymer whose main-chain repeating units contain the ester function and which may be used in the medical field. Notably, polyesters are understood to mean aliphatic polyesters such as poly(lactic acid) (PLA), poly(glycolic acid) (PGA), polycaprolactone (PCL), polybutyrolactone (PBL), polyhydroxyalkanoates (PHA), and copolymers thereof.

In a preferred embodiment, the polyester (block A) is selected from poly(lactic acid) (PLA), poly(glycolic acid) (PGA), polycaprolactone (PCL) and copolymers thereof.

Preferentially, the polyester present in the matrix is in a non-crosslinked form.

The poly(lactic acid) may be poly(L-lactic acid), poly(D-lactic acid) or poly(D,L-lactic acid). Advantageously, use is made of poly(D,L-lactic acid) (PDLLA). In this case, the polymer preferentially includes at least 50% L-lactic acid, and notably at least 60%, 70%, 75%, 80%, 85%, 90%, 95% or 99%. Specifically, by modifying the percentage of L-lactic acid relative to D-lactic acid, it is possible to act on the rate of degradation of the A and B block copolymer. An increase in the level of L-lactic acid makes it possible to slow down the rate of degradation of the copolymer. In certain embodiments of the invention, the composition comprises 100% PLLA as blocks A.

In the context of the invention, poly(oxyethylene) (PEO) is typically a linear polyether made from ethylene oxide or ethylene glycol monomers, preferably ethylene oxide monomers, Thus, according to the invention, block B may also be a polyethylene glycol (PEG) of high molecular weight greater than or equal to 50 kDa, notably having a molecular weight as defined below.

According to the invention, the poly(oxyethylene) (PEO) used for block B has a high molecular weight, so that the total molecular weight of the PEO in the copolymer is greater than or equal to 50 kDa.

In the context of the invention, the terms “molecular mass” and “molecular weight” are used interchangeably to refer to the weight average molecular mass (Mw), unless otherwise stated. According to the invention, the Mw is determined by size exclusion chromatography performed in dimethylformamide as the analytical solvent, using a poly(ethylene glycol) calibration range.

Advantageously, the total molecular mass of the PEO in the A and B block copolymer is between 50 kDa and 300 kDa. For example, the PEO blocks have a molecular mass of 50 kDa, 75 kDa, 95 kDa, 100 kDa, 125 kDa, 150 kDa, 200 kDa. 225 kDa, 250 kDa, 275 kDa or 300 kDa. In a particular embodiment, the PEO blocks used have a molecular mass of between 75 kDa and 150 kDa, preferentially between 80 kDa and 125 kDa, more preferentially between 90 kDa and 115 kDa, more preferably between 90 kDa and 110 kDa. In a particular embodiment, the PEO blocks used have a molecular mass of between 95 kDa and 105 kDa.

According to the invention, the PEO block used advantageously has an inherent viscosity of between 0.04 mg/ml and 0.6 mg/ml, preferentially between 0.08 mg/ml and 0.5 mg/ml, and even more preferably between 0.1 mg/ml and 0.3 mg/ml when measured with an Ubbelohde-type capillary viscometer at a concentration of 1 g/l, at 25° C. in chloroform.

Advantageously, the polymer matrix based on copolymers according to the invention comprises AB diblock, or ABA or BAB triblock copolymers, or mixtures thereof, notably [ABA and BAB], [AB and ABA], [AB and BAB], [ABA and BAB and AB]. Preferentially, the matrix comprises only A and B block copolymers. In a particular embodiment, the matrix according to the invention comprises only ABA or BAB triblock copolymers, and preferentially only ABA triblock copolymers. In a particular embodiment, the matrix according to the invention consists of ABA or BAB triblock copolymers, preferably ABA triblock copolymers.

According to the invention, in a matrix of AB and/or ABA copolymers, each PEO block has a molecular mass of greater than or equal to 50 kDa and advantageously between 50 kDa and 300 kDa, preferentially between 75 kDa and 150 kDa, preferably between 80 kDa and 125 kDa, more preferentially between 90 kDa and 115 kDa, more preferably between 90 kDa and 110 kDa, or even between 95 kDa and 105 kDa, whereas in a matrix comprising BAB copolymers, the sum of the molecular masses of the PEO blocks in said copolymer is greater than or equal to 50 kDa and advantageously between 50 kDa and 300 kDa, preferentially between 75 kDa and 150 kDa, preferably between 80 kDa and 125 kDa, more preferentially between 90 kDa and 115 kDa, even more preferably between 90 kDa and 110 kDa, or even between 95 kDa and 105 kDa.

In the context of the invention, the molar ratio represents the molar ratio of each of the repeating units (or units) of blocks A and B. As block B is PEO, the repeating units are ethylene oxides (“ethylene oxide unit” or EO), while the repeating units of block A (“ester unit”) are carboxylic acids such as lactic acid units. According to the invention, the EO/ester unit mole ratio in the copolymers of the matrix is between 0.5 and 5, and preferably between 1 and 3. The mole ratio is measured from the proton NMR (Nuclear Magnetic Resonance) spectrum in deuterated chloroform of the copolymer, in which the chemical shifts of the characteristic peaks of the PLA-PEO-PLA copolymers may be identified: CH (PLA): 5.1 ppm: CH₂ (PEO): 3.5 ppm: CH₃ (PLA): 1.5 ppm). Matrices whose copolymers have an EO/ester unit mole ratio, and notably an ethylene oxide/lactic acid unit (EO/LA) mole ratio, of between 1 and 3 have swelling and unfolding properties such that the volume increases up to tenfold in an aqueous or humid medium, making these matrices particularly suitable for intrauterine use. It has been found that polymer matrices with an EO/LA mole ratio of 3 degrade in a shorter time, in the order of 3 to 5 days, in an aqueous or humid medium such as phosphate-buffered saline PBS, than polymer matrices comprising an EO/LA mole ratio of 1, which degrade in a time of 5 to 20 days.

According to the invention, an “aqueous medium” refers to a medium with an osmolarity similar to the osmolarity of biological fluids. Phosphate-buffered saline (PBS) is commonly used as an aqueous medium, and is considered representative of biological fluids.

According to the invention, a “humid medium” refers to a medium equivalent to the aqueous medium, i.e. a medium with an osmolarity similar to the osmolarity of biological fluids, but the humid medium is non-liquid. The uterine cavity may be characterized as a non-liquid humid medium.

In a particular embodiment, the matrix consists of ABA triblock copolymers, where block A is PDLLA and block B is PEO with a molecular weight of between 90 kDa and 110 kDa, wherein the EO/LA mole ratio is 1.

In a particular embodiment, the matrix consists of ABA triblock copolymers, where block A is PDLLA and block B is PEO with a molecular weight of between 90 kDa and 110 kDa, wherein the EO/LA mole ratio is 2.

In another particular embodiment, the matrix consists of ABA triblock copolymers, wherein block A is PDLLA and block B is PEO with a molecular weight of between 90 kDa and 110 kDa, wherein the EO/LA mole ratio is 3.

The matrix according to the invention may be obtained via any method for synthesizing block copolymers that is known to those skilled in the art. For example, an ABA copolymer may be obtained by chain polymerization from the ends of block B. Typically, a lactide ring-opening polymerization initiated by the terminal hydroxyls of the PEO block is performed in the presence of a catalyst such as tin octanoate. This polymerization may be performed in the absence or presence of solvents. A BAB-type copolymer may be prepared, for example, by coupling methoxy-PEO onto a PLA chain whose two chain ends are carboxylic acid functions. Such a “difunctionalized” PLA is obtained, for example, by treating a PLA chain with succinic or adipic anhydride.

In a particular embodiment, the polymer matrix according to the invention may also comprise a PEG or PEO homopolymer, in particular a hydrophilic PEG or PEO homopolymer. Advantageously, the PEG or PEO homopolymer has a molecular mass of between 1 kDa and 300 kDa, preferably between 5 kDa and 200 kDa. In this embodiment, the homopolymer is not covalently bonded to the A and B block copolymers, nor to the haemostatic agent. Advantageously, the polymer matrix is based on 5% to 90% by mass, preferably from 10% to 80% by mass, of homopolymer, relative to the total mass of polymer matrix. For example, the polymer matrix may be based on 5%, 10%, 20%, 30%, 40%, 50%, 60%, 70%, 80% or 90% by mass of homopolymer, relative to the total mass of polymer matrix. According to this aspect, the polymer matrix is then based on 10% to 95% by mass, preferably 15% to 90% by mass, in particular 20% to 90% by mass, of A and B block copolymer, relative to the total mass of polymer matrix. The addition of a PEG or PEO homopolymer within the polymer matrix makes it possible to improve the swelling and unfolding properties of the system according to the invention, and notably to accelerate the swelling of the system after its introduction into the uterine cavity and thus to accelerate the contact of the system according to the invention with the walls of the uterine cavity. The mixing of the homopolymer with the matrix may be performed by any means known to those skilled in the art, for example by dissolution of copolymer and homopolymer powders in a common solvent (for example dichloromethane) followed by a solvent evaporation step, or by cold or hot mixing of homopolymer and copolymer powders (temperature of between 30° C. and 190° C.).

In a particular embodiment, the polymer matrix according to the invention may also comprise at least one super-disintegrant. For the purposes of the present invention, the term “super-disintegrant”, also referred to as “disintegrant”, means a solid compound which dissolves, swells or deforms rapidly in an aqueous or humid medium, which makes it possible to improve the permeability of a solid composition when it is placed in contact with an aqueous or humid medium. In particular, the super-disintegrant(s) are selected from modified starches (for example sodium starch glycolate), modified cellulose (for example croscarmellose sodium), crosslinked polyvinylpyrrolidones (for example crospovidone), or mixtures thereof. Advantageously, the polymer matrix is based on 5% to 90% by mass, in particular 10% to 85% by mass, in particular 10% to 80% by mass, more particularly 15% to 80% by mass, preferably 20% to 80% by mass, of super-disintegrant, relative to the total mass of polymer matrix. According to this aspect, the polymer matrix is then based on 10% to 95% by mass, preferably 15% to 90% by mass, in particular 20% to 90% by mass, of A and B block copolymer, relative to the total mass of polymer matrix. The addition of a super-disintegrant within the polymer matrix also makes it possible to improve the swelling and unfolding properties of the system according to the invention, and notably to accelerate the swelling of the system after it has been introduced into the uterine cavity and thus to accelerate the contact of the system according to the invention with the walls of the uterine cavity. The mixing of the super-disintegrant with the matrix may be performed by any means known to those skilled in the art, for example by cold or hot mixing of powders of super-disintegrant and matrix (temperature between 30° C. and 190° C.).

In another particular embodiment, the polymer matrix according to the invention may comprise a PEG or PEO homopolymer and at least one super-disintegrant as described above. In this embodiment, the polymer matrix is advantageously based on 10% to 95%, in particular 10% to 80%, by mass of A and B block copolymer, from 10% to 80% by mass of PEG or PEO homopolymer and from 10% to 80% by mass of super-disintegrant, relative to the total mass of polymer matrix.

In the context of the invention, the degradable intrauterine system for the release of a haemostatic agent into the uterine cavity comprises the polymer matrix based on A and B block copolymers as described above, optionally a PEG or PEO homopolymer and/or a super-disintegrant as described above, and a haemostatic agent, said haemostatic agent being intended to be released into the uterine cavity. Advantageously, the haemostatic agent/degradable polymer matrix mass ratio is between 0.0001% and 40%, preferably between 0.0001% and 30%, preferably between 0.0001% and 20%, preferably between 0.0001% and 10%, in particular between 0.0001% and 5%, in particular between 0.0001% and 1%, in particular between 0.0001% and 0.1%, in particular between 0.001% and 0.1%, more particularly between 0.005% and 0.05%.

In a preferred embodiment, in the system according to the invention, the haemostatic agent is not covalently bonded to the polymer matrix.

In the context of the present invention, the haemostatic agent is an active principle that promotes coagulation and stops the blood flow. The haemostatic agent according to the invention is advantageously selected from thrombin, tranexamic acid, calcium ions and gelatin, collagen, calcium alginate, fibrinogen, and oxidized cellulose. In a preferred embodiment, the haemostatic agent is thrombin.

In a particular embodiment, when the haemostatic agent is thrombin, the haemostatic agent/degradable polymer matrix mass ratio is advantageously between 0.0001% and 1%, preferably between 0.0001% and 0.1%, preferably between 0.001% and 0.1%, more particularly between 0.005% and 0.05%.

The preparation of the intrauterine system according to the invention may be performed by any means known to those skilled in the art, and notably by integrating the haemostatic agent during or after the forming of the polymer matrix.

According to a particular embodiment, the forming of the polymer matrix may be performed by any means known to those skilled in the art, for example by extrusion, solvent evaporation for example using dichloromethane, hot pressing, hot injection, electrospinning, moulding or 3D printing. The system according to the invention may then be obtained by adding the haemostatic agent to the interior of the polymer matrix by impregnating/swelling the polymer matrix in a solution or suspension comprising at least one haemostatic agent: or by coating the polymer matrix using a solution or suspension comprising at least one haemostatic agent, and optionally a water-soluble excipient, to form a coating on the surface of the polymer matrix: or by depositing on the surface of the polymer matrix a powder comprising a haemostatic agent and a water-soluble excipient and then hot pressing the polymer matrix and the powder, the surface of the polymer matrix being able to be coated with a solvent, such as acetone, ethanol or dichloromethane, to facilitate the attachment of the powder to the polymer matrix.

According to another particular embodiment, the system according to the invention may be prepared by integrating the haemostatic agent during the forming of the polymer matrix by means known to those skilled in the art, and notably via one of the following means:
impregnation/swelling of the polymers of the polymer matrix in a solution containing at least one haemostatic agent:
mixing dry powders of the polymers of the polymer matrix and haemostatic agent:
mixing by melting or softening of powders of polymers of the polymer matrix and haemostatic agent:
mixing a polymer solution and a haemostatic agent powder to give a suspension or a solution; and
mixing a solution of haemostatic agent and a powder of polymers of the polymer matrix to give a suspension or a solution.

The system according to the invention may then be formed by means known to those skilled in the art, and notably by hot pressing, hot injection, extrusion, solvent evaporation for example using dichloromethane, electrospinning, moulding or 3D printing, from the dispersion as obtained in the preceding step, comprising the haemostatic agent in the polymer matrix.

Thus, according to the invention, the haemostatic agent may be integrated into the very structure of the polymer matrix and/or may form an at least partial coating on the outer surface of the polymer matrix after forming.

Generally speaking, the thickness of the system according to the invention obtained depends on the amount of polymer matrix used and the surface of the support or mould used for the forming.

The system according to the invention may thus be in the form of a film, a tube, a porous structure, such as a 2D or 3D matrix of porous gel or hydrogel, etc.

A film is understood to be a two-dimensional material resulting, for example, from the evaporation on a flat surface of the solvent which dissolved the copolymer of the polymer matrix according to the invention. The thickness of such a film is advantageously between a few microns and several hundred microns, and notably between 10 μm and 1000 μm. In a particular embodiment, the film has a thickness of between 200 μm and 600 μm. The thickness is understood to be “dry”, in the sense that it is measured (for example by optical microscopy) under anhydrous conditions, after forming and optionally total evaporation of the solvent used for dissolving the copolymer.

The dimensions of the film may be adapted as needed, notably by cutting a larger film to the desired dimensions.

The films may be folded to form tubes or sleeves, held closed as needed by suturing or gluing, or folded in an accordion manner. The tubes may also be obtained directly by forming them around a cylinder or by extrusion.

In the context of the invention, the term “tube” refers to a three-dimensional hollow or solid cylindrical object whose walls are formed from a copolymer film comprising at least one A and B block copolymer according to the invention. Preferentially, the diameter of such a tube is several hundred microns, and notably between 300 μm and 3000 μm. In a particular embodiment, the tube has a wall thickness of 300 μm and a diameter of 900 μm.

In a particular embodiment, the system according to the invention is obtained by forming on a support intended to form a part of said system. For example, the material is dried on a woven or knitted textile made of another polymer, the whole thus forming a composite material.

Advantageously, the system according to the invention comprises only the constituents of the polymer matrix based on copolymers according to the invention, at least one haemostatic agent, and possibly traces of solvent. For example, the system comprises only PLA and PEO copolymers of high molecular weight (greater than or equal to 50 kDa), and at least one haemostatic agent. In one particular example, the system according to the invention comprises only PLA and PEO copolymers, wherein the PEO has a molecular weight of between 90 kDa and 100 kDa, and at least one haemostatic agent. In another particular example, the system according to the invention comprises only PLA and PEO copolymers, wherein the PEO has a molecular weight of between 90 kDa and 100 kDa, a PEO or PEG homopolymer and/or a super-disintegrant, and at least one haemostatic agent.

In certain cases, the system according to the invention may also include an additional additive or active principle, such as a therapeutic molecule such as an antibiotic. This additive or active principle may, for example, be added to the copolymer-based composition before or during the forming of the material, so as to be dispersed in the polymer matrix of the material. Alternatively, it is possible to impregnate or cover the material with this active principle after the forming. Preferentially, the additional active principle is capable of diffusing outwards from the material when it is in an aqueous or humid medium. By way of example, the additional active principle could be a vasoconstrictor.

The system according to the invention has swelling and unfolding properties that are particularly suitable for its intrauterine use. In particular, the specific properties of the intrauterine system according to the invention allow it to be easily introduced into the uterine cavity and then brought into contact with the uterine wall after swelling and unfolding. The swelling of the intrauterine system according to the invention in an aqueous or humid medium is particularly rapid, for example the degree of swelling within ten minutes after its insertion into the aqueous or humid medium having an osmotic pressure identical to that of biological fluids is between 150% and 450%. In addition, the increase in surface area achieved ten minutes after insertion into the aqueous or humid medium having an osmotic pressure identical to that of the biological fluids is between 50% and 200%. In addition, the swelling and contact with the uterine wall take place in a particularly short time, notably in two hours or less after introduction into the uterine cavity.

Advantageously, the material according to the invention has, in an aqueous or humid medium having an osmotic pressure identical to that of biological fluids, a degree of swelling after 24 hours of between 100% and 2000%, and preferably of between 150% and 1500%, relative to the surface area or swelling of the system before its insertion into the aqueous or humid medium having an osmotic pressure identical to that of biological fluids. The degree of swelling is measured as follows: a strip of dry material is weighed before being immersed for 24 hours at 37° C. in saline medium (PBS 1X) with agitation. After 24 hours, the excess PBS is removed with absorbent paper and the strip is reweighed. The degree of swelling is the ratio of the mass of the wet material strip to the mass of the dry material strip. The degree of swelling of the material is proportional to its percentage of water uptake, which corresponds to the ratio [(mass of the wet material strip-mass of the dry material strip)/mass of the dry material strip]×100.

The swelling of the material is accompanied by an increase in surface area and volume (“hydrated” surface area or volume) which is particularly advantageous in the case of medical use for the treatment of uterine haemorrhage, since it promotes the unfolding of the material in the uterine cavity, in particular for contact with the uterine wall.

The increase in surface area is very rapid and may notably reach 50% to 150% within a few minutes in an aqueous or humid medium, and notably between 50% and 200% after 10 minutes in an aqueous or humid medium with an osmotic pressure identical to that of biological fluids. The increase in surface area is accompanied by an increase in the volume of the material, which may be measured visually under the same conditions as for the measurement of the degree of swelling, by changing only the residence times in the saline solution. The increase in surface area corresponds to the ratio [(surface area of the “hydrated” strip after a time t of immersion-surface area of the “dry” strip)/surface area of the “dry” strip]×100.

The specific swelling properties of the intrauterine system according to the invention thus enable it to come rapidly into contact with the uterine wall and to release the haemostatic agent onto the uterine wall almost immediately.

Advantageously, the degradable intrauterine system according to the invention allows a release into the uterine cavity of at least 30% of the haemostatic agent initially present within 10 minutes or less after introduction of the system according to the invention into the uterine cavity. In particular, the degradable intrauterine system according to the invention advantageously allows a release into the uterine cavity of at least 30% of the haemostatic agent initially present within less than 8 minutes, 6 minutes, 5 minutes, 4 minutes, 3 minutes, 2 minutes, or 1 minute (limits included), after introduction of the system according to the invention into the uterine cavity.

Advantageously, the degradable intrauterine system according to the invention allows a release into the uterine cavity of at least 50%, preferably at least 60%, of the haemostatic agent initially present within 10 minutes or less after introduction of the system according to the invention into the uterine cavity. In particular, the degradable intrauterine system according to the invention advantageously allows a release into the uterine cavity of at least 50%, preferably at least 60%, of the haemostatic agent initially present within less than 8 minutes, 6 minutes, 5 minutes, 4 minutes, 3 minutes (limits included), after introduction of the system according to the invention into the uterine cavity.

In a preferred embodiment, the degradable intrauterine system according to the invention advantageously allows a release into the uterine cavity of:
at least 30% of the haemostatic agent initially present within 8 minutes or less:
at least 50% of the haemostatic agent initially present within 10 minutes or less; and/or
at least 80% of the haemostatic agent initially present within 30 minutes or less:
after introduction of the system according to the invention into the uterine cavity.

In a preferred embodiment, the degradable intrauterine system according to the invention advantageously allows a release into the uterine cavity of:
at least 50% of the haemostatic agent initially present within 6 minutes or less:
at least 60% of the haemostatic agent initially present within 10 minutes or less; and/or
at least 80% of the haemostatic agent initially present within 30 minutes or less:
after introduction of the system according to the invention into the uterine cavity.

In the context of the invention, the release profile of the haemostatic agent into the uterine cavity is measured according to the ELISA (Enzyme-Linked Immunosorbent Assay) method.

In the context of the invention, the rapid release of the haemostatic agent onto the uterine wall enables fast and efficient treatment of uterine haemorrhage.

An additional particularly advantageous feature of the system according to the invention is that it has anti-adhesion properties. Once haemostasis has been achieved, the cicatrization process follows its course and there is a risk that this may lead to the two walls of the uterus coming together in the form of a fibrous bridge known as an adhesion or synechia. The presence of a membrane or film with anti-adhesion properties makes it possible to create a physical and mechanical barrier between the walls, and thus allows cicatrization without synechiae.

An additional particularly advantageous feature of the system according to the invention is that it is degradable in an aqueous or humid medium. In particular, the system according to the invention degrades after a residence time in the uterine cavity of between 1 and 40 days, preferentially between 2 and 20 days, and more preferentially between 3 and 15 days, while retaining its integrity for a time of at least between 1 and 5 days. The degradation of the material is due to the progressive hydrolysis of the ester bonds of the polyester blocks followed by dissolution of the PEO-containing blocks. The loss of mechanical properties of the material is directly related to its degradation. The degradation may be evaluated by measuring the decrease in molecular weight of a strip of material over time, after immersion at 37° C. in saline medium (PBS IX) with stirring, for example by Size Exclusion Chromatography. It is also possible to evaluate the decrease in the dynamic viscosity of the material. The dissolution of the PEO blocks and hydrolysis of the polyester blocks in the uterine cavity is gradual, and is usually sufficient after 5 to 15 days to allow removal of the material. The degradation properties of the system according to the invention thus allow the system to remain intact in the uterine cavity for a time that is sufficient to treat uterine haemorrhage and then to degrade sufficiently to enable it to be eliminated naturally.

In a particular embodiment, the degradable intrauterine system according to the invention comprises at least one haemostatic agent and is in the form of a film or tube obtained by forming a polymer matrix consisting of ABA triblock copolymers where A is PDLLA and B is PEO with a molecular weight of between 90 kDa and 110 kDa, wherein the ratio EO/LA is 1.

In a particular embodiment, the degradable intrauterine system according to the invention comprises at least one haemostatic agent and is in the form of a film or tube obtained by forming a polymer matrix consisting of ABA triblock copolymers where A is PDLLA and B is PEO with a molecular weight of between 90 kDa and 110 kDa, wherein the ratio EO/LA is 2.

In another embodiment, the degradable intrauterine system comprises at least one haemostatic agent and is in the form of a film or tube obtained by forming a composition consisting of ABA triblock copolymers where A is PDLLA and B is PEO with a molecular weight of between 90 kDa and 110 kDa, wherein the ratio EO/LA is 3.

Advantageously, the haemostatic agent used in these embodiments is thrombin.

Advantageously, the degradable intrauterine system according to the invention is in the form of a film having a triangular or trapezoidal shape, so as to be in contact with the wall of the uterine cavity, once hydrated and unfolded by swelling.

The film may, for example, have a dry thickness of from 300 to 600 microns.

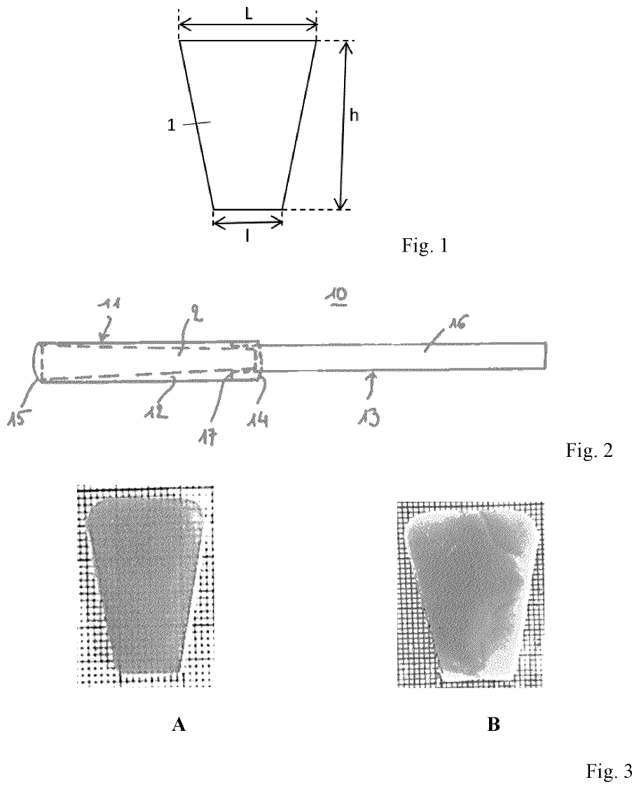

In a particular embodiment, as shown in FIG. 1, the film 1 has a trapezoidal shape with a height h of between about 1 and 4 cm, a largest width L of between about 1 and 2.5 cm, and a smallest width 1 of between about 0.5 and 1.5 cm. For example, the trapezoid has a height of about 2.5 cm, a largest width L of about 2 cm and a smallest width 1 of about 1 cm. These dimensions are readily adaptable by those skilled in the art, notably within the above ranges, according to the type of patient to be treated, depending on whether she is primiparous or multiparous, her age, the uterine anatomy, the reasons for fearing the occurrence of synechia, etc.

### The Haemostatic Agent

A subject of the invention is also a haemostatic agent for use in the treatment of uterine haemorrhage, wherein the haemostatic agent is delivered into the intrauterine cavity by means of a degradable intrauterine system according to the invention. The intrauterine system is thus as described above in the description.

A subject of the invention is also the use of a haemostatic agent for the preparation of a medicinal composition in the form of an intrauterine system according to the invention for the treatment of uterine haemorrhage. The intrauterine system is as described above in the description.

A subject of the invention is also a method for treating uterine haemorrhage in a patient in need thereof, comprising the administration into the uterine cavity of an intrauterine system according to the invention. The intrauterine system is as described above in the description.

In the context of the present invention, the haemostatic agent is advantageously selected from thrombin, tranexamic acid, calcium ions, gelatin, collagen, calcium alginate, fibrinogen, and oxidized cellulose. In a preferred embodiment, the haemostatic agent is thrombin.

### The Kit

A subject of the invention is also a kit comprising an intrauterine system according to the invention, and means for inserting the system into the uterine cavity. The kit according to the invention advantageously comprises means for inserting and placing the material in the uterine cavity.

For example, as shown in FIG. 2, the kit 10 according to the invention may comprise a hollow cylindrical inserter 11, in the bore 12 of which a film 2 having an inverted trapezoidal shape is housed. Advantageously, in order to minimize the dimensions of the inserter 11, the film is housed in the bore 12 in a compacted form. For example, the film is accordion-folded and held tightly by the inner walls of the bore 12. Only when released into the uterine cavity does the accordion unfold. This unfolding is further aided by the almost concomitant increase in volume of the film, the polymers of which swell with water on contact with the intrauterine fluid.

The kit 10 advantageously includes a plunger 13 mounted to slide in translation at a distal end 14 of the inserter, the opposite proximal end 15 being the end through which the inserter 11 is intended to be introduced into the uterine cavity. The plunger 13 consists of a rod 16 which, when pushed inside the bore 12 of the inserter 11, towards the proximal end 15, drives the film 2 in translation towards the outside of the inserter 11.

Advantageously, the plunger 13 comprises stop means 17 at the proximal end of the rod 16, said stop means 17 being intended to come up against the wall of the bore 11 bordering the proximal end 15 of the inserter, so as to inform the person handling the kit 10 that the film 2 has been completely ejected from the inserter and is in position in the uterine cavity. It then suffices to remove the insertion means/inserter assembly by simply pulling outwards, the film 2 itself remaining in position in the uterine cavity.

Such a kit allows the degradable intrauterine system according to the invention to be reliably introduced and put in place. Moreover, this compacted form enables the dimensions of the material to be even further reduced before swelling, which facilitates its introduction through the patient's cervix.

The kit according to the invention may notably be used in the case of patients suffering from uterine haemorrhage. The compacted form of the material and the use of a small applicator facilitate its insertion in the often sensitive uterine cavity of these patients. In addition, its natural elimination during the menstrual cycle avoids the need for an additional intervention on the patient by medical staff to remove said device.

The invention will now be illustrated with the aid of the examples below. These examples are merely presented as non-limiting indications of the invention.

### EXAMPLES

### Example 1: Preparation of a Degradable Intrauterine System for the Release of Thrombin into the Uterine Cavity

### 1. Synthesis of ABA Triblock Copolymers

a. Materials

Commercial poly(ethylene oxide) (PEO): Supplier Sigma-Aldrich, CAS No. 25322-68-3. Commercial PEO was analysed in the laboratory by Size Exclusion Chromatography (SEC) so as to determine its weight average molar mass (Mw). The analysis was performed in an analytical solvent (dimethylformamide), and the Mw was determined using a poly(ethylene glycol) calibration range. The weight average molar mass Mw is 95 000 Da and its inherent viscosity is 0.16 ml/mg.

Commercial D,L-lactide: supplier Corbion Purac, CAS No. 95-96-5.

b. Method

The ABA triblock is synthesized in the following manner:

PEO (Mw 95 000) (200 g) and D,L-lactide (458 g) are dried under vacuum at room temperature for 24 hours. PEO and D,L-lactide are introduced into a polymerization flask in the presence of tin octanoate (85 mg). 10 successive cycles of vacuum (10-3 bar) and inertization with argon are then performed. The mixture is then heated to 140° C. and a further 10 successive vacuum and argon-inertization cycles are performed. The mixture is returned to room temperature and then placed in an ice bath. Once crystallized, the reaction mixture is placed under dynamic vacuum for 30 min and then sealed under dynamic vacuum. The mixture is then placed in an oven with mechanical rotation at 140° C. for 3 days. The mixture is dissolved in dichloromethane and precipitated from an ether/ethanol mixture. The precipitate is recovered and then dried under vacuum for 24 hours.

c. Characterization

The final composition of the copolymer was determined by 1H NMR proton nuclear magnetic resonance and an EO/LA mole ratio of 3 is deduced.

Two-dimensional NMR analysis (DOSY) indicates that the synthesis did indeed result in an ABA triblock (PLA50-PEO-PLA50).

The copolymer was also analysed by Size Exclusion Chromatography (SEC) so as to determine its average molar mass Mw and its dispersity Ip.

With an analytical solvent such as dimethylformamide and using a poly(ethylene glycol) calibration range, an Mw of 123 000 Da and a dispersity in the region of 5 were obtained.

In addition, Thermogravimetric Analysis (TGA) made it possible to determine the degradation temperature of the copolymer, which is 256° C.

### 2. Forming of the Degradable Polymer Matrix (as a Film)

The ABA triblock is formed by hot pressing so as to form a film. 2.5 mg of powder are pressed between two plates heated to 85° C., for 9 minutes and under a pressure of 20 MPa. The film obtained, 500 μm thick, is then cut with a sample punch to obtain films having the following dimensions: Height 25 mm;

Large base 20 mm; Small base 10 mm.

### 3. Preparation of the Intrauterine Thrombin Release System According to the Invention

a. Materials

The prototype is prepared using thrombin of human origin, in lyophilized form, supplier Sigma-Aldrich (CAS No. 9002-04-4). The activity of the protein, included in the product's certificate of analysis, is as follows: >2000 NIH units/mg protein.

b. Forming of Prototype A

Polyethylene glycol (PEO) (homopolymer) with an average molar mass Mw of 100 kDa is dissolved in a saline solution (pH 6.5) with stirring at room temperature to obtain a concentration of 70 mg/mL. 1.4 mg of lyophilized thrombin powder (i.e. 33.6 μg of pure thrombin) are then dissolved in 750 μL of this solution. The PEO-thrombin solution thus obtained is placed in a freeze-dryer at −60° C. under 0.025 mbar for 16 hours. The resulting powder is weighed and then divided into two parts of equal mass. The powder is distributed evenly on both sides of the film using a trapezoidal template (PTFE sheet, thickness 0.5 mm, dimensions equivalent to those of the film). Finally, this assembly is pressed at 40° C. for 30 seconds, applying a pressure of 20 MPa. The prototype of the degradable intrauterine system in the form of a trapezoidal film includes:
200 mg of ABA copolymer
52.5 mg of PEO homopolymer (i.e. a mass percentage of PEO homopolymer in the polymer matrix of 20.79%)
33.6 μg of pure thrombin (i.e. a thrombin/degradable polymer matrix mass ratio of 0.0133%)
   c. Forming of prototype B

1.4 mg of lyophilized thrombin are dissolved in 70 μL of saline solution (pH 6.5). After stirring, the solution obtained is deposited on the film previously folded with a Finnpipette™ F1.

35 μL are deposited on one side of a film, in the hollows formed by the succession of folds. The film is left to stand at room temperature for 1 hour, so as to allow the absorption of the solution. In the same manner, the remaining 35 μL are deposited and absorbed on the second side of the folded film. The thrombin-impregnated film is then dried in a freeze-dryer at −60° C. under 0.025 mbar for 16 hours.

### 4. Evaluation of the Properties of the Intrauterine System According to the Invention

a. Swelling in a Model Used for the Release Test

The film is placed in a 50 ml bottle containing 10 ml of phosphate-buffered saline (pH 7.4). The bottle is placed at 37° C. under mechanical stirring (87 rpm). After 10 minutes, an increase in surface area of 79% relative to its initial surface area and an increase in its mass (by water uptake) of 188% relative to its initial mass are observed. FIG. 3 shows the dry film at TO (area 357 mm², mass 0.208 g) on the left (FIG. 3A); and the swollen film after 10 minutes (area 639 mm², mass 0.599 g) on the right (FIG. 3B).

b. Unfolding in a Uterine Cavity Model

The film obtained previously may be inserted into a uterine cavity model using an inserter.

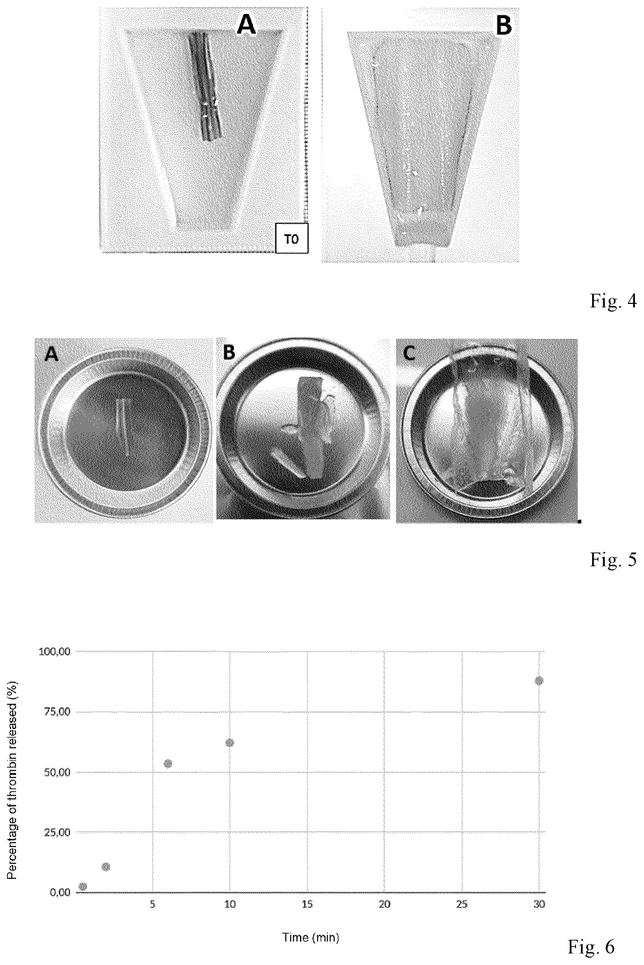

As seen in FIG. 4, the film unfolds and swells to cover the entire surface of the uterine cavity. FIG. 4A shows the film immediately after insertion into the uterine cavity model. FIG. 4B shows the film unfolded 2 hours after insertion into the uterine cavity.

c. Degradation

FIG. 5 next shows the degradation of the film a few days after insertion into the uterine cavity (FIG. 5A=film at T0). The film remains in the form of a film for 5 days, then gradually degrades into fragments (FIG. 5B), dissolves (FIG. 5C) and is totally evacuated through the cervix.

### 5. Evaluation of the Thrombin Release Kinetics

a. Methods
i. In-Vitro Release Method

The prototype is placed in a 50 ml bottle containing 10 ml of phosphate-buffered saline (pH 7.4). The bottle is placed at 37° C. under mechanical stirring (87 rpm). 2 μL of solution are withdrawn before the film is introduced at TO, then after introduction at 30 sec, 1 min, 2 min, 4 min, 6 min, 8 min and 10 min of release. Each withdrawn sample is analysed via the enzyme-linked immunosorbent assay (ELISA method). The amount of thrombin released is calculated for each sampling time from the equation of the calibration curve obtained with the standard range.

ii. Thrombin Assay Method

The ELISA test (supplied by Abcam: reference ab270210) used is designed for the quantitative measurement of thrombin. The thrombin (analyte) present in the solution collected during the release assay is captured by an uptake antibody, and by a detector antibody conjugated to a reporter, which will reveal the amount of thrombin present in the analysed sample. The whole complex (uptake antibody/analyte/detector antibody) is in turn immobilized by immunoaffinity of an anti-labelling antibody coating the well. A signal is thus generated, proportional to the amount of analyte (thrombin) bound to the antibody complex. The signal intensity is measured at 450 nm using a microplate reader.

iii. Calculation Method

Serial dilution is performed using a stock solution of thrombin (80 000 μg/mL, supplied in the ELISA kit) to obtain a concentration range from 0 to 9000 μg/mL. Optical density (OD) measurements at 450 nm, obtained for each concentration, are used to plot a calibration curve relating the optical density to the thrombin concentration. The equation of this curve will make it possible to determine the thrombin concentration of unknown samples. Then, knowing the amount of thrombin contained in the prototype, it is possible to evaluate the percentage of thrombin released as a function of time.

b. Results
i. Calibration Curve

The calibration curve is linear (r2=0.992) over the concentration range 0-9000 μg/mL. The same calibration curve was used to determine the concentration of thrombin in solution, released from prototype A.

ii. Prototype A

FIG. 6 and Table 1 show the percentage of thrombin released over time from prototype A. Under in-vitro release conditions, thrombin is released from the polymer film over time. The release of thrombin starts at about 30 seconds and at least 60% of the thrombin contained in the prototype is released after 10 minutes.

TABLE 1

| Time | Percentage of thrombin released relative to the |
|---|---|
| (minutes) | initial amount |
| 0.5 | 2 |
| 2 | 11 |
| 6 | 53 |
| 10 | 62 |
| 30 | 88 |

### 6. Evaluation of the Haemostatic Properties (In Vitro)

a. Materials
i. Prototypes

Prototypes A and B are formed using the method described in part 3.

ii. Haemostasis Support

Whole blood with CPD (citrate phosphate dextrose) is supplied by the Etablissement Français du Sang. Before being used for coagulation tests, this blood must be recalcified: to do this, calcium chloride (CaCl₂)-CAS 10043-52-4-supplied by Sigma-Aldrich) must be added to the blood. For the subsequent tests, 3 mL of CaCl₂) (concentration of 0.122 mol/L) are added to 30 mL of whole blood.

b. Method for Determining the Coagulation Time
i. In Vitro Model

30 mL of whole blood with CPD (unrecalcified), contained in a 50 mL Falcon tube, are heated on a water bath at 37° C. for 30 minutes prior to the start of the test. After addition of CaCl₂), 10 mL of the recalcified blood are introduced into a plastic uterine cavity model (dimensions: height 75 mm; large base 45 mm; small base 12 mm). This model is held upside down (small base/open end up) to prevent blood flow during the test. A prototype is then introduced into the model using an inserter. The whole assembly is placed in an oven at 37° C.

ii. Determination of the Coagulation Time

Coagulation is observed visually. The coagulation time is the time when all the blood contained in the uterine cavity model has set. To determine this, the model (containing the blood and the prototype) is rotated by 180° every minute for the first 10 minutes, then every 2 minutes until coagulation is observed. The tests are performed in triplicate: the average of the coagulation times obtained is then calculated.

c. Results
i. Controls

A control test is set up: a coagulation test on whole blood only (no prototype included, no haemostatic agent).

ii. Coagulation Test Results

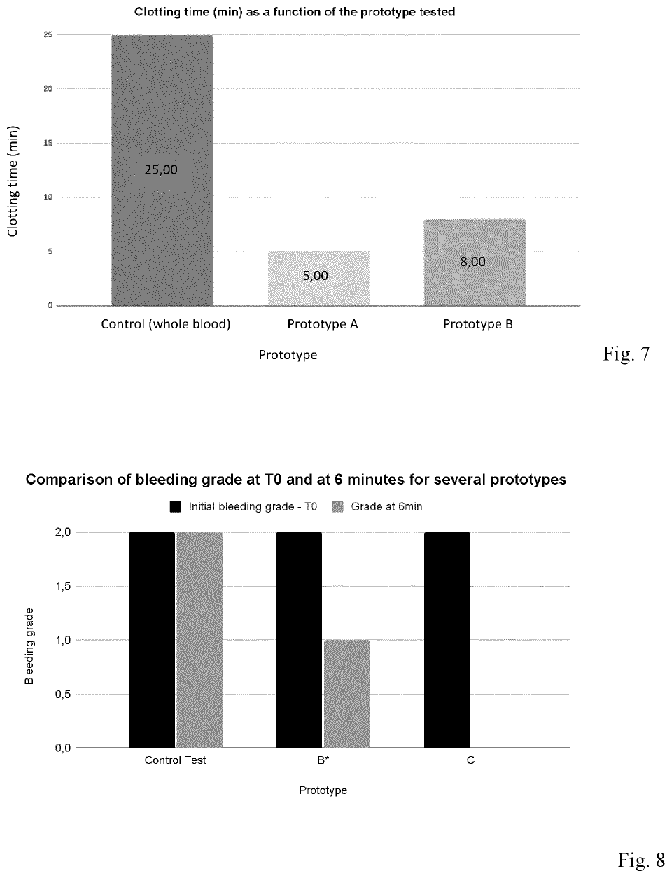

FIG. 7 shows the coagulation time (min) as a function of the prototypes or control tested. The biological activity of the released thrombin (in this case the coagulation activity) is obtained from 5 min onwards: the blood is partly absorbed by the film, which unfolds, swells and releases thrombin. The following observations may be made:
The coagulation time of whole blood in the absence of film and thrombin (control-whole blood) is 25 minutes:
The coagulation time of blood with prototype A is 5 minutes; and
The coagulation time of blood with prototype B is 8 minutes.

This system for thrombin release from a PLA50-PEO-PLA50 copolymer film thus significantly decreases the blood coagulation time in a uterine cavity model.

### 7. Evaluation of the Haemostatic Properties (In Vivo)

i. Bleeding Scale

A standardized semi-quantitative bleeding scale is used to evaluate bleeding. This bleeding scale will be used throughout the test to observe the evolution of the bleeding. See below the scale:
0=No bleeding
0.5=Ooze (blood observed at the edges, but not flowing)
1=Very slight bleeding (blood flows very slowly from the site)
2=Slight (blood flows slowly)
3=Moderate (blood flowing fast without pulsatile action)
4=Severe (blood flowing fast, pulsing and squirting out of wound)

A bleeding grade from 1 to 2 is preferred for this test.

ii. In-Vivo Model

The pig is selected due to its anatomical size and organ structure, which are similar to that of humans as well as similarity to humans with respect to the coagulation system.

A section is made on the liver and/or spleen of the pig, allowing to insert a prototype inside parenchyma. The hemostatic prototype will be in direct contact with the bleeding wound.

To obtain a grade of 1 to 2, the following section dimensions are used: 1 cm length: 0.5 cm in depth.

iii. Determination of Hemostatic Properties

The evolution of the bleeding grade is observed in order to evaluate whether the prototype is able to decrease the bleeding grade.

The initial bleeding is scored approximately 1 min after section creation, when the severity of the bleed is stable. The prototype is then inserted into the wound using forceps. The timer is triggered once the prototype is in place. Hemostasis performance (bleed score) is scored at 3 minimum time points (e.g. 1, 3 and 6 minutes) using the bleeding scale defined above. The test is stopped at 6 minutes.

A comparison between the initial bleeding grade and the bleeding grade at 6 minutes is performed to evaluate the hemostatic performance of the prototypes tested.

iv. Prototypes Preparation

Prototypes have been prepared following the same process as the one explained in section 3 above for prototype B (polymer film impregnated with thrombin solution). Squares of polymer sheets have been cut at the dimensions of the wounds (1×0.5 cm) and then impregnated with a thrombin solution:

Prototype B*: Polymer sheet sample cut 1×0.5 cm with thrombin spot in the middle, on both sides of the prototypes. Thrombin units: 13.3 NIH units. Composition: 99.35% copolymère ABA+0.65% Thrombin powder (0.015% pure thrombin).

Prototype C: Polymer sheet sample cut 1×0.5 cm with thrombin on the whole surface, on both sides of the prototypes. Thrombin units: 13.3 NIH units. Thrombin units: 26.7 NIH units. Composition: 98.75% copolymère ABA+1.25% Thrombin powder (0.03% pure thrombin).

### V. Results

The results are summarized in FIG. 8.

Of note, the bleeding scale is associated with the blood: if the bleeding scale is 0, there is hemostasis (no blood flow).

The following observations can be made:
At 6 minutes, the blood flow remains the same when there is nothing (Control Test) while the blood flow decreases by half (Prototype B*), or the bleeding stops completely (Prototype C) when a thrombin impregnated film sample is introduced into the wound.
When thrombin is distributed over the entire surface of the polymer sheet sample (Prototype C), in higher quantity, the hemostasis is faster; the distribution of thrombin should cover all the surfaces of the wound to achieve complete hemostasis.

### 8. Comparative Tests (In Vitro)

Comparative tests have been carried out to compare the technical effect of the molecular weight of the PEO in the block copolymers A and B.

Table 2 below lists all the block copolymers A and B (PLA-PEO-PLA) synthesized and used in the comparative solubility test, with the respective molecular weights of the PEO used.

TABLE 2

| Copolymers Composition | | | |
|---|---|---|---|
| PLA-PEO-PLA | Ratio EO/LA | PEO Molecular mass | |
| Test 1 | 1 | 6 | kDa |
| Test 2 | 3 | 6 | kDa |
| Test 3 | 5 | 6 | kDa |
| P3 | 1 | 100 | kDa |
| P4 | 3 | 100 | kDa |

From these copolymers, films were made by solvent evaporation. The films all have a thickness of between 250 and 300 μm. The copolymers and films were manufactured according to the methods described in example 1.

It has been very difficult to form films from copolymers Test 1, Test 2 and Test 3.

The films were then immersed in PBS (physiological buffer pH=7.4). Each film had an initial dry mass of 10 mg and the volume of incubation of PBS solution was 1 mL. The macroscopic changes of these films in PBS were compared.

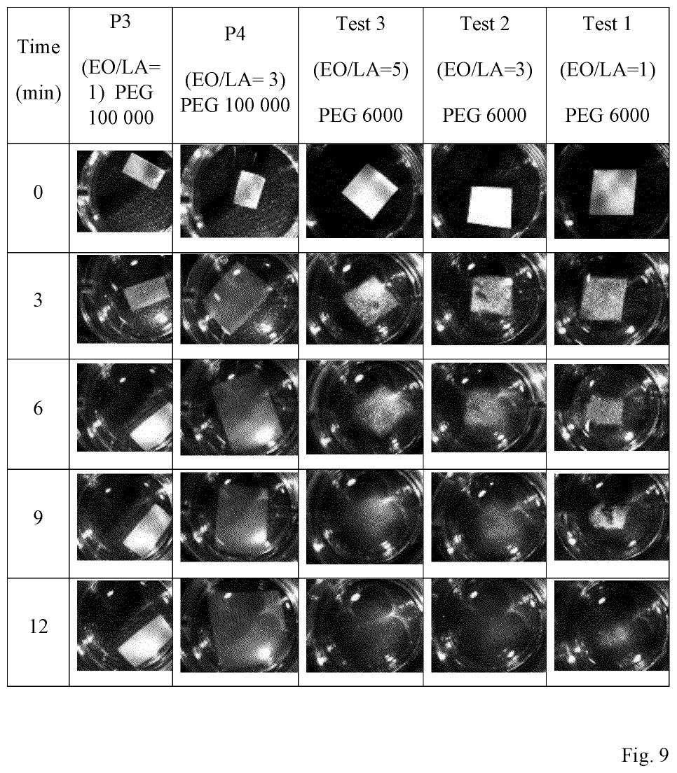

FIG. 9 shows the disintegration rate of each of these films after an incubation time in PBS between 0 and 12 minutes.

Both P3 and P4 films, whose copolymers comprise PEG 100,000 and have an EO/LA ratio between 1 and 3, increase in area but remain in the form of films during the period studied.

In contrast, the copolymer-based films comprising PEG 6,000 and having an EO/LA ratio of between 1 and 5 (namely Test 1, Test 2 and Test 3) have too much solubility (see FIG. 9, showing that such films disintegrate after only a few minutes in the water), rendering it unfit for any use in an aqueous medium and in particular a use for treating uterine haemorrhage. Consequently, the copolymer-based films comprising PEG 6,000 do not present the swelling and unfolding properties that are essential to treat uterine haemorrhage reliably and rapidly.

1-15. (canceled)

16. A degradable intrauterine system for the release of a haemostatic agent into the uterine cavity, comprising:
a degradable polymer matrix based on copolymers comprising at least one A and B block copolymer, wherein:
the A block is a polyester;
the B block is a poly(oxyethylene) (PEO);
the weight-average molecular mass of the blocks B is greater than or equal to 50 kDa; and
the ethylene oxide unit/ester unit mole ratio is between 0.5 and 5; and
at least one haemostatic agent for release into the uterine cavity.

17. The degradable intrauterine system according to claim 16, wherein the haemostatic agent is selected from thrombin, tranexamic acid, calcium ions, gelatin, collagen, calcium alginate, fibrinogen, and oxidized cellulose.

18. The degradable intrauterine system according to claim 17, wherein the haemostatic agent is thrombin.

19. The degradable intrauterine system according to claim 16, wherein the haemostatic agent/degradable polymer matrix mass ratio is between 0.01% and 40%.

20. The degradable intrauterine system according to claim 16, wherein the haemostatic agent is not covalently bonded to the polymer matrix.

21. The degradable intrauterine system according to claim 16, further comprising at least one polyethylene glycol (PEG) or poly(oxyethylene) (PEO) homopolymer, which is not covalently bonded to the polymer matrix.

22. The degradable intrauterine system according to claim 16, further comprising at least one super-disintegrant.

23. The degradable intrauterine device according to claim 22, wherein the super-disintegrant is selected from modified starches, modified celluloses and crosslinked polyvinylpyrrolidones.

24. The degradable intrauterine system according to claim 16, wherein at least 30% of the haemostatic agent initially present in the system is released within 10 minutes or less after introduction of the system into the uterine cavity.

25. The degradable intrauterine system according to claim 16, wherein at least 50% of the haemostatic agent initially present in the system is released within 10 minutes or less after introduction of the system into the uterine cavity.

26. The degradable intrauterine system according to claim 16, wherein the degradable polymer matrix is based on A and B block copolymers selected from AB diblock copolymers and ABA and BAB triblock copolymers, and mixtures thereof.

27. The degradable intrauterine system according to claim 16, wherein the weight-average molecular mass of the blocks B in the A and B block copolymer is between 75 kDa and 150 kDa.

28. The degradable intrauterine device according to claim 27, wherein the weight-average molecular mass of the blocks B in the A and B block copolymer is: a) between 80 and 125 kDa, b) between 90 and 115 kDa, or c) between 90 kDa and 110 kDa.

29. The degradable intrauterine system according to claim 16, wherein the ethylene oxide unit/ester unit ratio of the A and B block copolymer is from 1 to 3.

30. The degradable intrauterine system according to claim 16, wherein the blocks A of the A and B block copolymer are poly(lactic acids).

31. The degradable intrauterine device according to claim 30, wherein the poly(lactic acids) are selected from poly(L-lactic acid), poly(D-lactic acid) and poly(D,L-lactic acid).

32. The degradable intrauterine system according to claim 16, wherein the degradable polymer matrix has a degree of swelling of between 150% and 450% after 10 minutes in an aqueous or humid medium having an osmotic pressure identical to that of biological fluids.

33. A method of treating uterine haemorrhage comprising the administration of a degradable intrauterine system according to claim 16 into the uterine cavity.

34. A kit comprising an intrauterine system according to claim 16 and means for inserting the system into the uterine cavity.
